(19) United States
(12) Patent Application Publication
(10) Pub. No.: US 2020/0054486 A1
(43) Pub. Date: Feb. 20, 2020

(54) BIODEGRADABLE INTRAVITREAL IMPLANTS

(71) Applicant: Tathagata DUTTA, BERHAMPORE (IN)

(72) Inventor: Tathagata DUTTA, BERHAMPORE (IN)

(21) Appl. No.: 16/487,824

(22) PCT Filed: Sep. 26, 2017
(86) PCT No.: PCT/IN2017/050426

§ 371 Date: Aug. 21, 2019

(30) Foreign Application Priority Data
Jul. 4, 2017 (IN) 201741023455

(51) Int. Cl. A61F 9/00; A61K 47/30

(52) U.S. Cl. CPC A61F 9/0017; A61K 9/0051; A61K 47/30

(57) ABSTRACT

A biodegradable intravitreal implant comprising an antioxidant and a biodegradable polymer that releases the antioxidant at a predetermined rate effective to sustain release of an amount of the antioxidant from the implant for a prolonged period of time after the implant is placed into the vitreous of an eye.

### TECHNICAL FIELD

This invention relates to drug delivery dosage forms/systems and methods to treat medical conditions of the eye. Specifically, this invention relates to intravitreal implant drug delivery dosage forms/systems of antioxidants for drug delivery within the eye.

### BACKGROUND ART

Age elated macular degeneration (ARMD) is a severe problem of the eye which accounts for the loss of vision of huge number of elderly population across the globe. The disease which starts as a mild innocent problem of the eye, including occasional floaters and black dots in front of the eye gradually progresses to the loss of peripheral vision to complete loss of vision.

Antioxidants are well known to slow down the progression of the ARMD disease. There are several formulations available in the market containing single or combination of antioxidants like Alpha Tocopherol, Lutein, Zeaxanthin, Selenium, etc for oral administration for slowing down the progression of the disease. There are several drawbacks with orally administered drugs or bioactives as the drug does not reach the eye at appropriate concentrations and has either none or very poor pharmacological action in the eye when administered through oral route.

Hence, there is a need for a topical formulation of antioxidants like Lutein and/or Zeaxanthin for Intraocular or Intravitreal administration that will release the drug at a predetermined rate for an extended period of time thereby maintaining a steady concentration of antioxidants in the vitreous humor for a prolonged period of time thereby precluding the need of repeated administration and leading to better safety and efficacy, for effective management of ARMD.

### SUMMARY OF INVENTION

Accordingly, the invention provides new topical drug delivery dosage forms that releases the active agent at a predetermined rate effective to sustain release of the said active agent to achieve the desired therapeutic effects, and methods of making such dosage forms. The topical drug delivery dosage form of the invention is a biodegradable intravitreal implants. The biodegradable intravitreal implant of the invention when placed in the vitreous of an eye releases the active agent at a predetermined rate effective to give sustained release for a prolonged time.

The biodegradable intravitreal implants according to the disclosure herein comprise an antioxidant and a biodegradable polymer. The antioxidant according to the disclosure herein is a carotenoid selected from lutein, zeaxanthin or a combination thereof. According to the disclosure herein the biodegradable polymer is selected from homopolymers of lactic acid, and copolymers of lactic acid and glycolic acid, i.e., poly(lactide-co-glycolide) or polylactide or “PLGA” polymers, which includes polymers of lactic acid alone, copolymers of lactic acid and glycolic acid, mixtures of such polymers, mixtures of such copolymers, and mixtures of such polymers and copolymers—the lactic acid being either in racemic or in optically active form.

Alternatively the polymer in the biodegradable intravitreal implants of the disclosure herein include ethyl cellulose, cellulose acetate, cellulose acetate propionate, cellulose butyrate, cellulose propionate, cellulose valerate, cumaroneindene polymer, dibutylaminohydroxypropyl ether, ethylene-vinyl acetate copolymer, glycerol distearate, hydroxypropylmethyl cellulose phthalate, 2-methyl-5-vinylpyridine methacrylate-methacrylic acid copolymer, polyamino acids, polyanhydrides, polycaprolactone, polycarbonate, polybutadiene, polyesters, aliphatic polyesters, polybutadiene, polyesters, polyhydroxybutyric acid, polymethyl methacrylate, polymethacrylic acid ester, polyolesters or polypropylene, vinylchloride-propylene-vinlyacetate copolymer, vinylchloride-vinylacetate polymer, polyvinyl acetal diethylamino acetate, polyvinyl acetate, polyvinyl alcohol, polyvinyl butyral or polyvinly formal, dendrimers such as Polymethyl methacrylate dendrimers, Poly L Lysine dendrimers or Poly propyleneimine dendrimers, polysaccharides such as alginic acid, chitin, chitosan, chondroitin, dextrin, dextran, proteins such as albumin, casein, collagen, fibrin, fibrinogen, gelatin or hemoglobin; waxes such as whale wax, bee wax, paraffin wax, castor wax and so forth, and higher lipid acids such as myristic acid, palmitic acid, stearic acid, behenic acid, phospholipids such as phosphatidyl choline, phosphatidylserine, polyethylene glycol derivatives of phospholipids like PEG-Phosphatidylcholine or a combination thereof.

The biodegradable intravitreal implants according to the disclosure herein comprises antioxidant in a range of about 30% by weight to about 70% by weight of the implant, and the biodegradable polymer in a range of about 30% by weight to about 70% by weight of the implant.

The biodegradable intravitreal implants according to the disclosure herein is prepared by
a. dissolving antioxidant in an organic solvent,
b. separately dissolving the polymer in an organic solvent,
c. then mixing the solution of the antioxidant and the solution of the polymer under a high speed homogenizer and poured into 1-2% of Polyvinyl alcohol solution in water for injection and homogenizing the same to prepare an emulsion,
d. the emulsion is then sterilized by aseptic filtration, the sterile emulsion is then homogenized aseptically using a high pressure homogenizer, several cycles, till the desired particle size for the ocular implant is obtained, and
e. the ocular implant is then washed several times with water for injection to remove traces of the poly vinyl alcohol and then dried aseptically and packed.

The organic solvent is selected from the group comprising of methylene chloride (dicholoromethane), ethanol, methanol, chloroform, dimethyl sulfoxide , N-hexane, cyclohexane, acetone or a combination thereof.

### DESCRIPTION OF EMBODIMENTS

As described herein, the disclosure provides biodegradable intravitreal implants comprising an antioxidant and a biodegradable polymer that releases the antioxidant at a predetermined rate effective to provide sustained release of the antioxidant from the implant for 3 to 12 months after the implant is placed into the vitreous of an eye, wherein the antioxidant comprises from about 30% by weight to about 70% by weight of the implant, and the biodegradable polymer comprises from about 30% by weight to about 70% by weight of the implant.

The biodegradable intravitreal implants according to the disclosure herein comprises an antioxidant and a biodegradable polymer. The antioxidant according to the disclosure herein is a carotenoid selected from lutein, zeaxanthin or a combination thereof. According to the disclosure herein the biodegradable polymer is selected from homopolymers of lactic acid, and copolymers of lactic acid and glycolic acid, i.e., poly(lactide-co-glycolide) or polylactide or “PLGA” polymers or derivatives thereof or a cellulose derivative or a dendrimer or a polysaccharide or a protein or vinylchloride-propylene-vinlyacetate copolymer, vinylchloride-vinylacetate polymer, or a wax or a higher lipid acid or a phospholipid or a combination thereof. Preferably, the biodegradable polymer is polylactides, polyglycolides PLGA or mixture thereof.

The biodegradable polymers suitable for use in the biodegradable intravitreal implants of the disclosure herein include, but not limited to, homopolymers of lactic acid, and copolymers of lactic acid and glycolic acid, i.e., poly(lactide-co-glycolide) or polylactide or “PLGA” polymers, which includes polymers of lactic acid alone, copolymers of lactic acid and glycolic acid, mixtures of such polymers, mixtures of such copolymers, and mixtures of such polymers and copolymers—the lactic acid being either in racemic or in optically active form. The ratio of lactic acid residues to glycolic acid residues can vary; ethyl cellulose, cellulose acetate, cellulose acetate propionate, cellulose butyrate, cellulose propionate, cellulose valerate, cumaroneindene polymer, dibutylaminohydroxypropyl ether, ethylene-vinyl acetate copolymer, glycerol distearate, hydroxypropylmethyl cellulose phthalate, 2-methyl-5-vinylpyridine methacrylate-methacrylic acid copolymer, polyamino acids, polyanhydrides, polycaprolactone, polycarbonate, polybutadiene, polyesters, aliphatic polyesters, polybutadiene, polyesters, polyhydroxybutyric acid, polymethyl methacrylate, polymethacrylic acid ester, polyolesters or polypropylene, vinylchloride-propylene-vinlyacetate copolymer, vinylchloride-vinylacetate polymer, polyvinyl acetal diethylamino acetate, polyvinyl acetate, polyvinyl alcohol, polyvinyl butyral or polyvinly formal, dendrimers such as Polymethyl methacrylate dendrimers, Poly L Lysine dendrimers or Poly propyleneimine dendrimers, polysaccharides such as alginic acid, chitin, chitosan, chondroitin, dextrin, dextran, proteins such as albumin, casein, collagen, fibrin, fibrinogen, gelatin or hemoglobin; waxes such as whale wax, bee wax, paraffin wax, castor wax and so forth, and higher lipid acids such as myristic acid, palmitic acid, stearic acid, behenic acid, phospholipids such as phosphatidyl choline, phosphatidylserine, polyethylene glycol derivatives of phospholipids like PEG-Phosphatidylcholine.

The biodegradable intravitreal implants according to the disclosure herein is prepared by

a. dissolving the antioxidant in an organic solvent,

b. separately dissolving the polymer in an organic solvent,

c. then mixing the solution of the antioxidant and the solution of the polymer under a high speed homogenizer and poured into 1-2% of Polyvinyl alcohol solution in water for injection and homogenizing the same to prepare an emulsion,

d. the emulsion is then sterilized by aseptic filtration,

e. the sterile emulsion is then homogenized aseptically using a high pressure homogenizer, several cycles, till the desired particle size for the ocular implant is obtained, and

f. the ocular implant is then washed several times with water for injection to remove traces of the poly vinyl alcohol and then dried aseptically and packed.

The organic solvent is selected from the group comprising of methylene chloride (dicholoromethane), ethanol, methanol, chloroform, dimethyl sulfoxide, N-hexane, cyclohexane, acetone or a combination thereof. Most preferably the organic solvent is methylene chloride.

The method for preparing the biodegradable intravitreal implants according to the disclosure described herein is not limited to the above method. The biodegradable intravitreal implants according to the disclosure described herein can be prepared by using various other techniques. The other methods that can be used to prepare the biodegradable intravitreal implants as described herein include, but are not limited to, extrusion methods, co-extrusion methods, solvent evaporation methods, phase separation methods, interfacial methods, molding methods, injection molding methods, carver press method, die cutting methods, heat compression or combinations thereof.

In one embodiment, the biodegradable implants comprise the antioxidants lutein and zeaxanthin and a polymer Poly (D,L-lactide-co-glycolide) PLGA.

### EXAMPLES

### Example 1

TABLE 1
Lutein + Zeaxanthin intravitreal implant

| S. No | Ingredient | mg/mL | |
|---|---|---|---|
| 1 | Lutein | 600 | mcg |
| 2 | Zeaxanthin | 300 | mcg |
| 3 | Poly (D,L-lactide-co-glycolide) PLGA | 0.200-750 | mg |

Lutein and Zeaxanthin are dissolved in methylene chloride and, separately the polymer is dissolved in methylene chloride. Both the solution of the Polymer and the Lutein (active agent) and Zeaxanthin (active agent) are mixed under a high speed homogenizer and poured into 1-3% of Polyvinyl alcohol solution in water for injection and homogenized to prepare an emulsion. The emulsion is aseptically filtered through 0.45 micron filter, followed by 0.22 micron. The sterile material so obtained is homogenized aseptically using a high pressure homogenizer, several cycles, till the desired particle size is obtained. The intravitreal implants so obtained, washed several times with water for injection to remove traces of the poly vinyl alcohol and then dried aseptically and packed.

In another embodiment, the biodegradable intravitreal implants comprise an antioxidant lutein and a polymer Poly (D,L-lactide-co-glycolide) PLGA.

### Example 2

TABLE 2
Lutein intravitreal implant

| S. No | Ingredient | mg/mL | |
|---|---|---|---|
| 1 | Lutein | 600 | mcg |
| 2 | Poly (D,L-lactide-co-glycolide) PLGA | 0.200-300 | mg |

Lutein is dissolved in methylene chloride and, separately the polymer is dissolved in methylene chloride. Both the solution of the Polymer and the Lutein (active agent) are mixed under a high speed homogenizer and poured into 1-3% of Polyvinyl alcohol solution in water for injection and homogenized to prepare an emulsion. The emulsion is aseptically filtered through 0.45 micron filter, followed by 0.22 micron. The sterile material so obtained is homogenized aseptically using a high pressure homogenizer, several cycles, till the desired particle size is obtained. The intravitreal implants so obtained, washed several times with water for injection to remove traces of the poly vinyl alcohol and then dried aseptically and packed.

In another embodiment of the invention, the biodegradable intravitreal implants comprise an antioxidant zeaxanthin and a polymer Poly (D,L-lactide-co-glycolide) PLGA.

### Example 3

TABLE 3
Zeaxanthin intravitreal implant

| S. No | Ingredient | mg/mL | |
|---|---|---|---|
| 1 | Zeaxanthin | 600 | mcg |
| 2 | Poly (D,L-lactide-co-glycolide) PLGA | 0.200-300 | mg |

Zeaxanthin is dissolved in methylene chloride and, separately the polymer is dissolved in methylene chloride. Both the solution of the Polymer and the Zeaxanthin (active agent) are mixed under a high speed homogenizer and poured into 1-3% of Polyvinyl alcohol solution in water for injection and homogenized to prepare an emulsion. The emulsion is aseptically filtered through 0.45 micron filter, followed by 0.22 micron. The sterile material so obtained is homogenized aseptically using a high pressure homogenizer, several cycles, till the desired particle size is obtained. The intravitreal implants so obtained, washed several times with water for injection to remove traces of the poly vinyl alcohol and then dried aseptically and packed.

According to another embodiment, the biodegradable intravitreal implants comprise the antioxidants lutein and zeaxanthin and a polymer Poly (D,L-lactide-co-glycolide) PLGA.

### Example 4

TABLE 4
Lutein + Zeaxanthin intravitreal implant

| S. No | Ingredient | mg/mL |
|---|---|---|
| 1 | Lutein | 600 mcg |
| 2 | Zeaxanthin | 300 mcg |
| 3 | Poly Lactic acid (PLA) | 0.150-300 |

Lutein and Zeaxanthin are dissolved in methylene chloride and, separately the polymer is dissolved in methylene chloride. Both the solution of the Polymer and the Lutein (active agent) and Zeaxanthin (active agent) are mixed under a high speed homogenizer and poured into 1-2% of Polyvinyl alcohol solution in water for injection and homogenized to prepare an emulsion. The emulsion is aseptically filtered through 0.45 micron filter, followed by 0.22 micron. The sterile material so obtained is homogenized aseptically using a high pressure homogenizer, several cycles, till the desired particle size is obtained. The intravitreal implants so obtained, washed several times with water for injection to remove traces of the poly vinyl alcohol and then dried aseptically and packed.

According to another embodiment, the biodegradable intravitreal implants comprise the antioxidants lutein and zeaxanthin and a polymer Poly-L-Lysine Dendrimer.

### Example 5

TABLE 5
Lutein + Zeaxanthin intravitreal implant

| S. No | Ingredient | mg/mL |
|---|---|---|
| 1 | Lutein | 90 mcg |
| 2 | Zeaxanthin | 90 mcg |
| 3 | Poly-L-Lysine Dendrimer | 0.3-300 |

Lutein and Zeaxanthin are dissolved in methylene chloride and, separately the polymer is dissolved in water for injection. Both the solution of the Polymer and the Lutein (active agent) and Zeaxanthin (active agent) are mixed under a high speed homogenizer and poured into 1-2% of Polyvinyl alcohol solution in water for injection and homogenized to prepare an emulsion. The emulsion is aseptically filtered through 0.45 micron filter, followed by 0.22 micron. The sterile material so obtained is homogenized aseptically using a high pressure homogenizer, several cycles, till the desired particle size is obtained. The intravitreal implants so obtained, washed several times with water for injection to remove traces of the poly vinyl alcohol and then dried aseptically and packed.

According to yet another embodiment, the biodegradable intravitreal implants comprise the antioxidants lutein and zeaxanthin and a polymer Hydroxy Propyl Methyl Cellulose (HPMC).

### Example 6

TABLE 6
Lutein + Zeaxanthin intravitreal implant

| S. No | Ingredient | mg/mL | |
|---|---|---|---|
| 1 | Lutein | 90 | mcg |
| 2 | Zeaxanthin | 90 | mcg |
| 3 | Hydroxy Propyl Methyl Cellulose (HPMC) | 0.03-30 | mg |

Lutein and Zeaxanthin are dissolved in methylene chloride and, separately the HPMC is dissolved in water for injection. Both the solution of the Polymer and the Lutein (active agent) and Zeaxanthin (active agent) are mixed under a high speed homogenizer and poured into 1% of Polyvinyl alcohol solution in water for injection and homogenized to prepare an emulsion. The emulsion is aseptically filtered through 0.45 micron filter, followed by 0.22 micron. The sterile material so obtained is homogenized aseptically using a homogenizer, several cycles, to obtain a micro emulsion which is then aseptically poured into molds of appropriate size and allowed to dry. The intravitreal implants so obtained were then dried aseptically and packed.

According to another embodiment, the biodegradable intravitreal implants comprise the antioxidants lutein and zeaxanthin and a polymer Phosphatidylcholine.

### Example 7

TABLE 7
Lutein + Zeaxanthin intravitreal implant

| S. No | Ingredient | mg/mL | |
|---|---|---|---|
| 1 | Lutein | 90 | mcg |
| 2 | Zeaxanthin | 90 | mcg |
| 3 | Phosphatidylcholine | 0.03-30 | mg |

Lutein and Zeaxanthin are dissolved in diethylether and, separately the phospholipid is dissolved in diethylether. Both the solution of the phospholipid and the Lutein (active agent) and Zeaxanthin (active agent) are mixed under a high speed homogenizer. The solution is aseptically filtered through 0.45 micron filter, followed by 0.22 micron. The sterile material so obtained is then aseptically poured into molds of appropriate size and allowed to dry. The intravitreal implants so obtained were then dried aseptically and packed.

1. A biodegradable intravitreal implant comprising an antioxidant and a biodegradable polymer that releases the antioxidant at a predetermined rate effective to provide sustained release of the antioxidant from the implant for 3 to 12 months after the implant is placed into the vitreous of an eye, wherein the antioxidant comprises of about 30% by weight to about 70% by weight of the implant, and the biodegradable polymer comprises of about 30% by weight to about 70% by weight of the implant.

2. The biodegradable intravitreal implant of claim 1, wherein the antioxidant is lutein or zeaxanthin or a combination thereof.

3. The biodegradable intravitreal implant of claim 1, wherein the biodegradable polymer is selected from the group comprising of homopolymers of lactic acid, and copolymers of lactic acid and glycolic acid, i.e., poly(lactide-co-glycolide) or polylactide or “PLGA” polymers, which includes polymers of lactic acid alone, copolymers of lactic acid and glycolic acid, mixtures of such polymers, mixtures of such copolymers, and mixtures of such polymers and copolymers—the lactic acid being either in racemic or in optically active form.

4. The biodegradable intravitreal implant of claim 1, wherein the biodegradable polymer is selected from the group comprising of ethyl cellulose, cellulose acetate, cellulose acetate propionate, cellulose butyrate, cellulose propionate, cellulose valerate, cumaroneindene polymer, dibutylaminohydroxypropyl ether, ethylene-vinyl acetate copolymer, glycerol distearate, hydroxypropylmethyl cellulose phthalate, 2-methyl-5-vinylpyridine methacrylate-methacrylic acid copolymer, polyamino acids, polyanhydrides, polycaprolactone, polycarbonate, polybutadiene, polyesters, aliphatic polyesters, polybutadiene, polyesters, polyhydroxybutyric acid, polymethyl methacrylate, polymethacrylic acid ester, polyolesters, polypropylene, vinylchloride-propylene-vinlyacetate copolymer, vinylchloride-vinylacetate polymer, polyvinyl acetal diethylamino acetate, polyvinyl acetate, polyvinyl alcohol, polyvinyl butyral or polyvinyl formal.

5. The biodegradable intravitreal implant of claim 1, wherein the biodegradable polymer is selected from dendrimers comprising of Polymethyl methacrylate dendrimers, Poly L Lysine dendrimers, Poly propyleneimine dendrimers; or polysaccharides comprising of alginic acid, chitin, chitosan, chondroitin, dextrin or dextran; or proteins comprising of albumin, casein, collagen, fibrin, fibrinogen, gelatin or hemoglobin; or waxes comprising of whale wax, bee wax, paraffin wax or castor wax; or higher lipid acids comprising of myristic acid, palmitic acid, stearic acid or behenic acid; or phospholipids comprising of phosphatidyl choline, phosphatidylserine, Polyethylene Glycol derivatives of Phospholipids like PEG-Phosphatidylcholine.

6. The biodegradable intravitreal implant of claim 1, wherein the biodegradable polymer is polylactides, polyglycolides PLGA or mixture thereof.

7. The biodegradable intravitreal implant of claim 1, wherein the biodegradable intravitreal implant is prepared by the method comprising the steps of:
a. dissolving the antioxidant in an organic solvent,
b. separately dissolving the polymer in an organic solvent,
c. then mixing the solution of the antioxidant and the solution of the polymer under a high speed homogenizer and poured into 1-2% of Polyvinyl alcohol solution in water for injection and homogenizing the same to prepare an emulsion,
d. the emulsion is then sterilized by aseptic filtration,
e. the sterile emulsion is then homogenized aseptically using a high pressure homogenizer through several cycles till the desired particle size for the ocular implant is obtained, and
f. the ocular implant is then washed several times with water for injection to remove traces of the poly vinyl alcohol and then dried aseptically.

8. The biodegradable intravitreal implant of claim 1, wherein the organic solvent is selected from the group comprising of methylene chloride (dicholoromethane), ethanol, methanol, chloroform, dimethyl sulfoxide, N-hexane, cyclohexane, acetone or a combination thereof.
